(19) United States
(12) Patent Application Publication
(10) Pub. No.: US 2026/0041398 A1
(43) Pub. Date: Feb. 12, 2026

(54) SYSTEM AND METHOD FOR DISPLAYING DATA QUALITY INFORMATION FOR ULTRASOUND IMAGING AND SWEEP GUIDANCE INFORMATION FOR ULTRASOUND DATA ACQUISITION

(71) Applicant: GE Precision Healthcare LLC, Waukesha, WI (US)

(72) Inventors: Amalie EKSTRAND, Copenhagen (DK); Nicolas BØTKJÆR, Ballerup (DK); Anders OLIN, Copenhagen (DK); Bo MARTINS, Rodovre (DK); Mads ANDERSEN, Copenhagen (DK)

(21) Appl. No.: 18/796,937

(22) Filed: Aug. 7, 2024

(51) Int. Cl. A61B 8/00

(52) U.S. Cl. CPC A61B 8/4281; A61B 8/429; A61B 8/461; A61B 8/469

(57) ABSTRACT

Various systems are provided for displaying data quality information for ultrasound imaging and sweep guidance information for ultrasound data acquisition. Ultrasound data of a region of interest of a subject may be acquired. An image of the region of interest of the subject may be generated. A region of the image that was generated using particular ultrasound data, of the ultrasound data, that includes a data quality that is less than a data quality threshold may be determined using an artificial intelligence (AI) model. The image may be displayed including a delineation of the region of the image that was generated using the particular ultrasound data. Furthermore, accurate registration in multiple locations of a big organ, such as the liver, is being assisted by ultrasound sweep guidance at the locations of interest as identified in a pre-operative imaging modality.

### TECHNICAL FIELD

The present disclosure relates to systems and methods for ultrasound imaging. More specifically, the present disclosure relates to a system and method for displaying data quality information for ultrasound imaging, and a system and method for displaying sweep guidance information for ultrasound data acquisition.

### BACKGROUND

The data quality of ultrasound data may significantly affect the viability of applications or procedures that utilize the ultrasound data. For example, an ultrasound imaging system may generate an ultrasound image using acquired ultrasound data, and display the ultrasound image. If the data quality of the ultrasound data is low, the ultrasound image might not accurately, or completely, depict a region of interest. As another example, an ultrasound imaging system may register ultrasound data with preoperative imaging data of another imaging modality. If the data quality of the ultrasound data is low, the ultrasound imaging system might not accurately, of adequately, register the ultrasound data with the preoperative imaging data. As another example, the ultrasound imaging system may track an interventional device during an interventional procedure, and display the tracked interventional device. If the data quality of the ultrasound data is low and image information such as an indication of a needle in the image is used at least as part of the tracking algorithm, the ultrasound imaging system might not accurately track or display the interventional device. As yet another example, the ultrasound imaging system may segment an anatomical structure from the ultrasound data, and display a three-dimensional (3D) image of the anatomical structure. If the data quality of the ultrasound data is low, the ultrasound imaging system might not accurately, or adequately, segment the anatomical structure.

In the foregoing situations, a reviewing entity (e.g., a physician, a sonographer, a clinician, etc.) might have difficulty deciphering a displayed image or have difficulty tracking a displayed interventional device. The foregoing technical problems may be exacerbated in situations where the ultrasound imaging system is being utilized during an intraoperative procedure because patient safety and procedural efficacy may rely on the accuracy and robustness of the underlying ultrasound data.

In other cases, a user might have difficulty in acquiring ultrasound data of sufficient data quality. For example, the user might have difficulty assessing the particular location at which to initiate scanning, the particular orientation of the ultrasound probe to use for scanning, the particular direction in which to move the ultrasound probe during scanning, the particular velocity at which to move the ultrasound probe during scanning, or the like. The foregoing technical problems might be exacerbated in situations where ultrasound data of a particular region of interest is to be acquired. Accordingly, if the data quality of the ultrasound data is low, then the user might be required to perform another scan which might introduce delay during interventional procedures.

Registration of ultrasound data during surgery to preoperative data of some imaging modality is a difficult task. One problem is organ deformations from the preoperative setup to the surgery setup. The deformation may be caused by different patient positioning, insufflation of carbon dioxide for laparoscopic surgery, by opening of the patient in case of open surgery, or by the surgery itself. For large organs, such as the liver, registration needs to be accurate at those positions of the organ where a lesion is seen on the preoperative imaging modality. For minimally invasive surgery, the ultrasound probe cannot be moved freely, and it is therefore useful to indicate to the user where and possibly how to sweep in order to acquire a sufficient amount of ultrasound data at the approximate location of each lesion to perform accurate registration at that location. The algorithm that creates this visual indication to the user is based on a coarse alignment between preoperative data and ultrasound as its input.

Coarse alignment can be established by holding a tracked probe in a specific position and orientation that is well-defined on the preoperative imaging modality. The coarse alignment may be used as input to the sweep guidance algorithm.

Visual indication of where to sweep is also useful if the quality or length of some original scan proves to be inadequate for the registration algorithm to function properly.

### SUMMARY

This summary introduces concepts that are described in more detail in the detailed description. It should not be used to identify essential features of the claimed subject matter, nor to limit the scope of the claimed subject matter.

In an aspect, a device may include a probe including: a transducer configured to transmit ultrasound signals towards a region of interest, and receive echo signals from the region of interest; a matching layer configured to have an acoustic impedance between the region of interest to be imaged and a material of the transducer; and a damping block configured to absorb ultrasound energy; a memory configured to store instructions; and one or more processors configured to execute the instructions to: acquire ultrasound data of the region of interest of a subject; generate an image of the region of interest of the subject based on the ultrasound data; determine, using an artificial intelligence (AI) model, a region of the image that was generated using particular ultrasound data, of the ultrasound data, that includes a data quality that is less than a data quality threshold; and display the image including a delineation of the region of the image that was generated using the particular ultrasound data, of the ultrasound data, that includes the data quality that is less than the data quality threshold.

In another aspect, a method may include acquiring ultrasound data of a region of interest of a subject; generating an image of the region of interest of the subject based on the ultrasound data; determining, using an artificial intelligence (AI) model, a region of the image that was generated using particular ultrasound data, of the ultrasound data, that includes a data quality that is less than a data quality threshold; and displaying the image including a delincation of the region of the image that was generated using the particular ultrasound data, of the ultrasound data, that includes the data quality that is less than the data quality threshold.

In another aspect, a non-transitory computer-readable medium may store instructions that, when executed by one or more processors, cause the one or more processors to: acquire ultrasound data of a region of interest of a subject; generate an image of the region of interest of the subject based on the ultrasound data; determine, using an artificial intelligence (AI) model, a region of the image that was generated using particular ultrasound data, of the ultrasound data, that includes a data quality that is less than a data quality threshold; and display the image including a delineation of the region of the image that was generated using the particular ultrasound data, of the ultrasound data, that includes the data quality that is less than the data quality threshold.

In yet another aspect of the invention, in order to perform registration between a preoperative imaging modality and live ultrasound, a method performs a visual indication guiding the user to position a tracked ultrasound probe at a specific point in space and/or at a specific orientation, or to perform an ultrasound sweep from a desired start position to a desired end position.

### BRIEF DESCRIPTION OF DRAWINGS

FIG. 1 is a diagram of an example system for displaying data quality information for ultrasound imaging and sweep guidance information for ultrasound data acquisition.

FIG. 2 is a diagram of example components of one or more systems of FIG. 1.

FIG. 3 is a diagram of an example ultrasound imaging system.

FIG. 4 is a diagram of an example tracking system.

FIG. 5 is a diagram of a preoperative imaging system.

FIG. 6 is a diagram of an example process for training an AI model.

FIG. 7 is a flowchart of an example process for displaying data quality information in association with ultrasound images.

FIG. 8 is a diagram of an example process for displaying data quality information for ultrasound imaging.

FIGS. 9A and 9B are diagrams of example ultrasound images displaying data quality information.

FIG. 10 is a flowchart of an example process for displaying data quality information in association with images.

FIG. 11 is a diagram of an example 3D image displaying data quality information.

FIG. 12 is a flowchart of an example process for displaying sweep guidance information for ultrasound data acquisition.

FIG. 13 is a diagram of sweep guidance information including a visual indicator for guiding an orientation and a velocity of an ultrasound probe during ultrasound data acquisition.

FIG. 14 is a diagram of sweep guidance information including a visual indicator for guiding a position of an ultrasound probe during ultrasound data acquisition.

FIG. 15 is a diagram of sweep guidance information including a visual indicator for guiding a movement of an ultrasound probe to reacquire ultrasound data corresponding to a region of a 3D image that was generated using ultrasound data that includes a data quality that is less than a data quality threshold.

FIG. 16 is a diagram of sweep guidance information including a visual indicator for guiding a position and an orientation of the ultrasound probe.

FIG. 17 is a diagram of sweep guidance information including a visual indicator for guiding a position and an orientation of the ultrasound probe to acquire ultrasound data.

FIG. 18 is a diagram of sweep guidance information including a visual indicator for guiding a position of the ultrasound probe to acquire ultrasound data.

FIG. 19 is a diagram of sweep guidance information including a visual indicator for guiding a position and an orientation of the ultrasound probe to acquire ultrasound data.

FIG. 20 is a diagram of sweep guidance information including a visual indicator for guiding a position and an orientation of the ultrasound probe to acquire ultrasound data.

### DETAILED DESCRIPTION

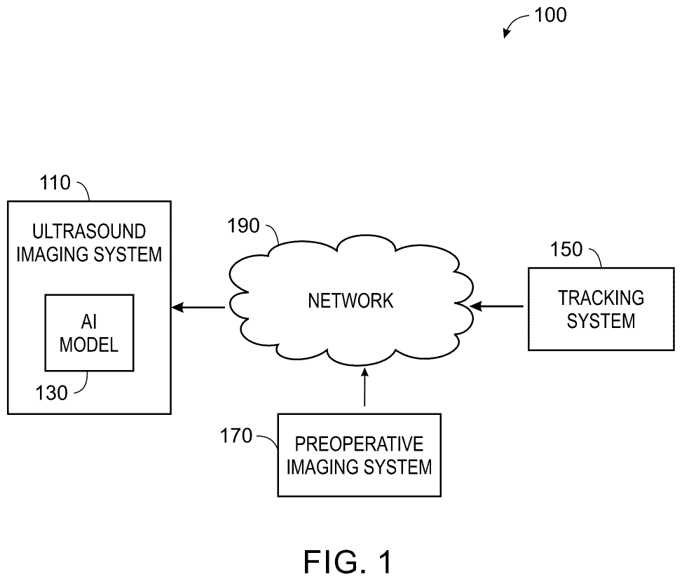

FIG. 1 is a diagram of an example system for displaying data quality information for ultrasound imaging and sweep guidance information for ultrasound data acquisition. As shown in FIG. 1, the system 100 may include an ultrasound imaging system 110, an artificial intelligence (AI) model 130, a tracking system 150, a preoperative imaging system 170, and a network 190.

The ultrasound imaging system 110 may be configured to acquire ultrasound data, and generate a medical image based on the ultrasound data. For example, the ultrasound imaging system 110 may be a two-dimensional (2D) ultrasound system, a 3D ultrasound system, a four-dimensional (4D) ultrasound system, a Doppler ultrasound system, or the like. According to an embodiment, the medical image may be an ultrasound image. According to another embodiment, the medical image may be a 3D image of an anatomical structure that is generated using ultrasound data. The anatomical structure may be a blood vessel, a tissue, an organ, or the like.

The AI model 130 may be configured to receive ultrasound data and determine a data quality of the ultrasound data. For example, the AI model 130 may be a decision tree (e.g., a classification tree, a regression tree, or the like), a linear regression model, a neural network (e.g., a deep neural network (DNN), a convolutional neural network (CNN), a recurrent neural network (RNN), or the like), a logistic regression model, a support vector machine, or the like.

The tracking system 150 may be configured to acquire tracking data corresponding to a tracked instrument. For example, the tracking system 150 may be an electromagnetic tracking system, an optical tracking system, an acoustic tracking system, an inertial tracking system, or the like. The tracked instrument may be an ultrasound probe, an interventional device (e.g., a catheter, a needle, or the like), or the like.

The preoperative imaging system 170 may be configured to acquire preoperative imaging data. For example, the preoperative imaging system 170 may be a computed tomography (CT) system, a magnetic resonance imaging (MRI) system, an ultrasound system, an X-ray system, a positron emission tomography (PET) device, or the like.

The network 190 may be configured to permit communication between the ultrasound imaging system 110, the tracking system 150, and the preoperative imaging system 170. For example, the network 190 may be a local area network (LAN), a wide area network (WAN), a metropolitan area network (MAN), a cellular network, a private network, an ad hoc network, an intranet, the Internet, a fiber optic-based network, or the like, and/or a combination of these or other types of networks.

The number and arrangement of the systems of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

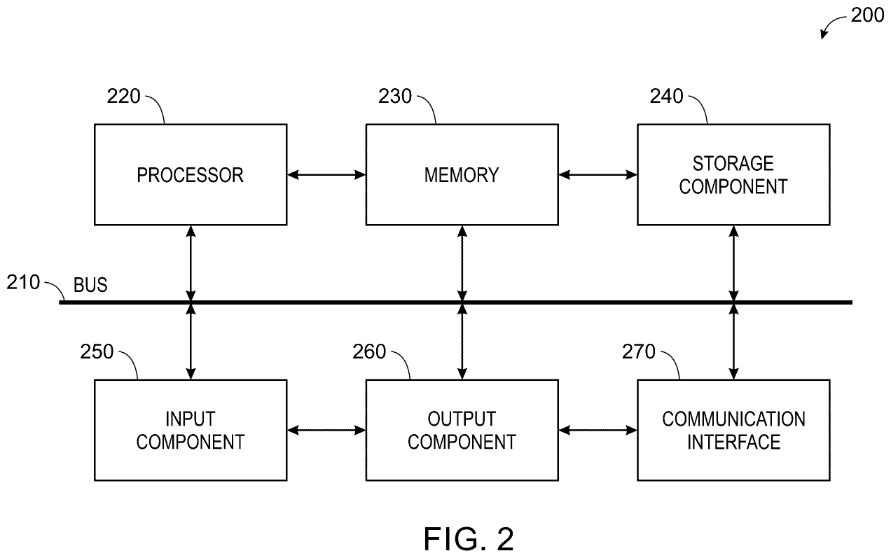

FIG. 2 is a diagram of example components of a system 200. The system 200 may correspond to the ultrasound imaging system 110, the intraoperative imaging system 130, the tracking system 150, and/or the preoperative imaging system 170. As shown in FIG. 2, the system 200 may include a bus 210, a processor 220, a memory 230, a storage component 240, an input component 250, an output component 260, and a communication interface 270.

The bus 210 includes a component that permits communication among the components of the system 200. The processor 220 may be implemented in hardware, firmware, or a combination of hardware and software. The processor 180 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 180 may include one or more processors capable of being programmed to perform a function. The processor 180 may include one or more processors 180 configured to perform the operations described herein. For example, a single processor 180 may be configured to perform all of the operations described herein. Alternatively, multiple processors 180, collectively, may be configured to perform all of the operations described herein, and each of the multiple processors 180 may be configured to perform a subset of the operations descried herein. For example, a first processor 180 may perform a first subset of the operations described herein, a second processor 180 may be configured to perform a second subset of the operations described herein, etc.

The memory 230 may include a random access memory (RAM), a read only memory (ROM), and/or another type of dynamic or static storage device (e.g., a flash memory, a magnetic memory, and/or an optical memory) that stores information and/or instructions for use by the processor 180.

The storage component 240 may store information and/or software related to the operation and use of the system 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the system 200 to receive information, such as via user input (e.g., a touch screen display, a keyboard, a keypad, a mouse, a button, a switch, a camera, and/or a microphone). Additionally, or alternatively, the input component 250 may include a sensor for sensing information (e.g., a global positioning system (GPS) component, an accelerometer, a gyroscope, and/or an actuator). The output component 260 may include a component that provides output information from the system 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the system 200 to communicate with other systems, such as via a wired connection, a wireless connection, or a combination of wired and wireless connections. The communication interface 270 may permit the system 200 to receive information from another system and/or provide information to another system. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The system 200 may perform one or more processes described herein. The system 200 may perform these processes based on the processor 180 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another system via the communication interface 270. When executed, the software instructions stored in the memory 230 and/or the storage component 240 may cause the processor 180 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the system 200 may include additional components, fewer components, different components, or differently arranged components than those shown in FIG. 2. Additionally, or alternatively, a set of components (e.g., one or more components) of the system 200 may perform one or more functions described as being performed by another set of components of the system 200.

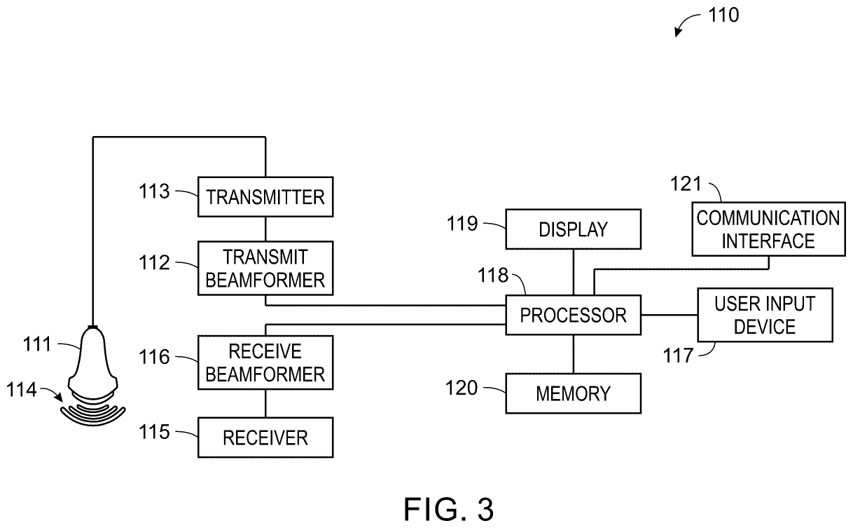

FIG. 3 is a diagram of an example ultrasound imaging system. As shown in FIG. 3, the ultrasound imaging system 110 may include an ultrasound probe 111, a transmit beamformer 112, a transmitter 113, elements 114, a receiver 115, a receive beamformer 116, a user input device 117, a processor 118, a display 119, a memory 120, and a communication interface 121. The foregoing components may be connected via wired or wireless connections.

The ultrasound probe 111 may be configured to acquire ultrasound data. For example, the ultrasound probe 111 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 111 may be configured to generate ultrasound signals, emit the ultrasound signals towards a region of interest of a subject, receive echo ultrasound signals that are back-scattered from the region of interest of the subject, generate ultrasound data based on the echo ultrasound signals, and output the ultrasound data. The region of interest may be any region of the anatomy of a subject. The subject may be a person, an animal, a phantom, or the like.

According to an embodiment, the ultrasound probe 111 may include a transducer configured to transmit ultrasound signals towards a region of interest, and receive echo signals from the region of interest, a matching layer configured to have an acoustic impedance between the region of interest to be imaged and a material of the transducer; and a damping block configured to absorb ultrasound energy.

The transmit beamformer 112 may be configured to apply delay times to electrical signals provided to the elements 114 to focus corresponding ultrasound signals at the region of interest. The transmitter 113 may be configured to transmit electrical signals to the elements 114 to drive the elements 114 to emit ultrasound signals towards the region of interest. The elements 114 may be configured to receive the electrical signals from the transmitter 113, convert the electrical signals into ultrasound signals, and emit the ultrasound signals towards the region of interest. The elements 114 may be configured to receive echo ultrasound signals that are back-scattered by the region of interest, convert the echo ultrasound signals into electrical signals, and provide the electrical signals to the receiver 115. The receiver 115 may be configured to receive electrical signals from the elements 114, and provide the electrical signals to the receive beamformer 116. The receive beamformer 116 may apply delay times to the electrical signals received from the elements 114.

The user input device 117 may be configured to receive a user input, and provide the user input to the processor 118. For example, the user input device 117 may be a touch screen display, a keyboard, a keypad, a mouse, a button, a switch, a microphone, or the like. Additionally, or alternatively, the user input device 117 may be configured to sense information. For example, the user input device 117 may sense information from an electro-magnetic positioning system, an inertial measurement system, an accelerometer, a gyroscope, an actuator, or the like.

The processor 118 may be configured to perform the operations as described herein. For example, the processor 118 may be a CPU, a GPU, an APU, a microprocessor, a microcontroller, a DSP, an FPGA, an ASIC, or the like. The processor 118 may be implemented in hardware, firmware, or a combination of hardware and software. The processor 118 may include one or more processors 118 configured to perform the operations described herein. For example, a single processor 118 may be configured to perform all of the operations described herein. Alternatively, multiple processors 118, collectively, may be configured to perform all of the operations described herein, and each of the multiple processors 118 may be configured to perform a subset of the operations described herein. For example, a first processor 118 may perform a first subset of the operations described herein, a second processor 118 may be configured to perform a second subset of the operations described herein, etc.

The processor 118 may be configured to control the ultrasound probe 111 to acquire ultrasound data. The processor 118 may be configured to control which of the elements 114 are active, and control the shape of a beam emitted from the ultrasound probe 111. The processor 118 may generate ultrasound images for display. For example, the processor 118 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 119 may be configured to display information. For example, the display 119 may be a monitor, an LED display, a cathode ray tube, a projector display, a touchscreen, tablet computer, mobile phone, or the like. The display 119 may display ultrasound images based on the ultrasound data in real-time. For example, the display 119 may display the ultrasound images within one second, two seconds, five seconds, etc., of the ultrasound data being acquired by the ultrasound probe 111.

The memory 120 may be configured to store information and/or instructions for use by the processor 118. The memory 120 may be a non-transitory computer-readable medium. For example, the memory 120 may be a RAM, a ROM, a flash memory, a magnetic memory, an optical memory, or the like. The memory 120 may be configured to store instructions that, when executed by the processor 118, cause the processor 118 to perform the operations described herein.

The communication interface 121 may be configured to enable the processor 118 to communicate with other systems, such as via a wired connection, a wireless connection, or a combination of wired and wireless connections. For example, the communication interface 121 may include an Ethernet interface, an optical interface, a coaxial interface, an infrared interface, an RF interface, a USB interface, a Wi-Fi interface, a cellular network interface, or the like.

The number and arrangement of the components of the ultrasound imaging system 110 shown in FIG. 3 are provided as an example. In practice, the ultrasound imaging system 110 may include additional components, fewer components, different components, or differently arranged components than those shown in FIG. 3. Additionally, or alternatively, a set of components (e.g., one or more components) of the ultrasound imaging system 110 may perform one or more functions described as being performed by another set of components of the intraoperative imaging system 130.

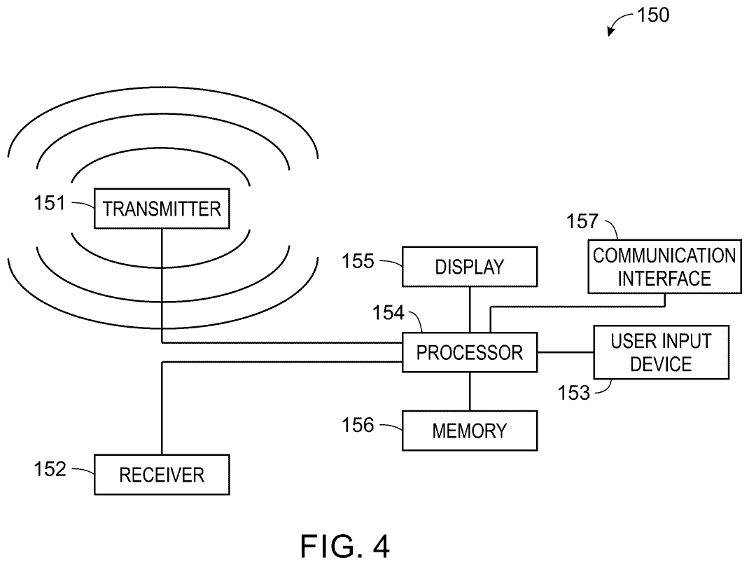

FIG. 4 is a diagram of an example tracking system 150. As shown in FIG. 4, the tracking system 150 may be an electromagnetic tracking system, and may include a transmitter 151, a receiver 152, a user input device 153, a processor 154, a display 155, a memory 156, and a communication interface 157.

The transmitter 151 may be configured to generate a magnetic field. The receiver 152 may be configured to output a signal in response to the magnetic field generated by the transmitter 151. The processor 154 may receive the output signal from the receiver 152, and acquire tracking data that identifies a position and/or an orientation of the receiver 152. The receiver 152 may be attached to, integrated with, provided in, etc., a tracked instrument. For example, according to an embodiment, the receiver 152 may be attached to the ultrasound probe 111 to track a position and/or an orientation of the ultrasound probe 111. Alternatively, the receiver 152 may be attached to a wand or other hand-held device to track a position and/or an orientation of a wand or the another hand-held device. Alternatively, the receiver 152 may be attached to an interventional device to track a position and/or an orientation of the interventional device. The interventional device may be a catheter, a needle, or the like.

The user input device 153 may be configured to receive a user input, and provide the user input to the processor 154. For example, the user input device 153 may be a touch screen display, a keyboard, a keypad, a mouse, a button, a switch, a microphone, or the like. Additionally, or alternatively, the user input device 153 may be configured to sense information. For example, the user input device 153 may sense information from an electro-magnetic positioning system, an inertial measurement system, an accelerometer, a gyroscope, an actuator, or the like.

The processor 154 may be configured to perform the operations as described herein. For example, the processor 154 may be a CPU, a GPU, an APU, a microprocessor, a microcontroller, a DSP, an FPGA, an ASIC, or the like. The processor 154 may be implemented in hardware, firmware, or a combination of hardware and software. The processor 154 may include one or more processors 154 configured to perform the operations described herein. For example, a single processor 154 may be configured to perform all of the operations described herein. Alternatively, multiple processors 154, collectively, may be configured to perform all of the operations described herein, and each of the multiple processors 154 may be configured to perform a subset of the operations described herein. For example, a first processor 154 may perform a first subset of the operations described herein, a second processor 154 may be configured to perform a second subset of the operations described herein, etc.

The processor 154 may be configured to control the transmitter 151 to acquire tracking data. The processor 154 may be configured to control excitations of the transmitter 151 to generate a magnetic field. The processor 154 may acquire tracking data based on controlling the transmitter 151.

The display 155 may be configured to display information. For example, the display 155 may be a monitor, an LED display, a cathode ray tube, a projector display, a touchscreen, tablet computer, mobile phone, or the like. The display 155 may display the tracking data in real-time. For example, the display 155 may display the tracking data within one second, two seconds, five seconds, etc., of the tracking data being acquired.

The memory 156 may be configured to store information and/or instructions for use by the processor 154. The memory 156 may be a non-transitory computer-readable medium. For example, the memory 156 may be a RAM, a ROM, a flash memory, a magnetic memory, an optical memory, or the like. The memory 156 may be configured to store instructions that, when executed by the processor 154, cause the processor 154 to perform the operations described herein.

The communication interface 157 may be configured to enable the processor 154 to communicate with other systems, such as via a wired connection, a wireless connection, or a combination of wired and wireless connections. For example, the communication interface 157 may include an Ethernet interface, an optical interface, a coaxial interface, an infrared interface, an RF interface, a USB interface, a Wi-Fi interface, a cellular network interface, or the like.

The number and arrangement of the components of the tracking system 150 shown in FIG. 4 are provided as an example. In practice, the tracking system 150 may include additional components, fewer components, different components, or differently arranged components than those shown in FIG. 4. Additionally, or alternatively, a set of components (e.g., one or more components) of the tracking system 150 may perform one or more functions described as being performed by another set of components of the tracking system 150.

Although FIG. 4 depicts the tracking system 150 as being an electromagnetic tracking system, it should be understood that the embodiments herein are applicable to other types of tracking systems, such as optical tracking systems, acoustic tracking systems, ultrasound tracking systems, or the like.

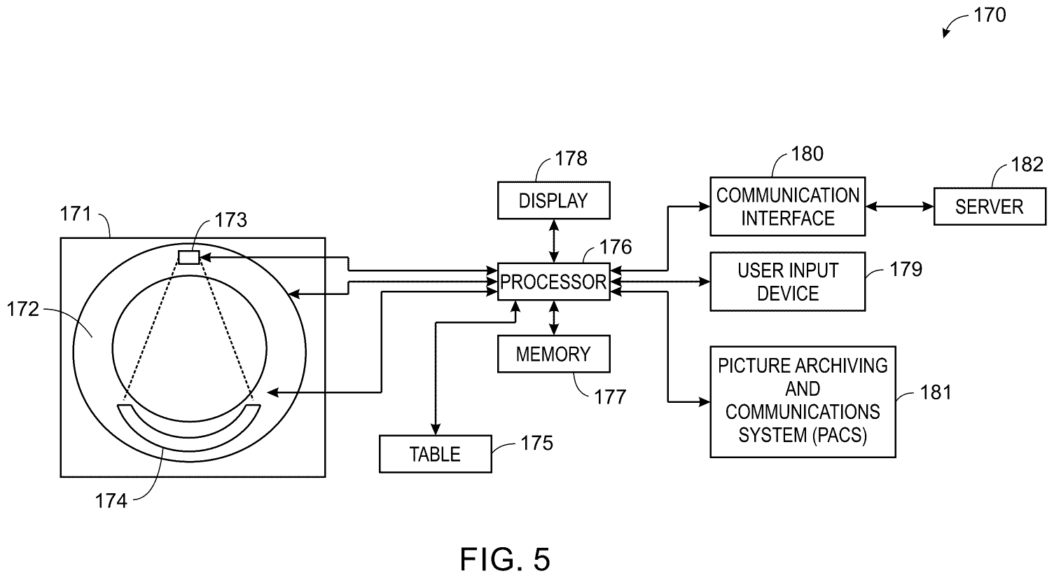

FIG. 5 is a diagram of a preoperative imaging system 170. As shown in FIG. 5, the preoperative imaging system 170 may be a CT imaging system, and may include a gantry 171, a rotational frame 172, an X-ray source 173, an X-ray detector 174, a table 175, a processor 176, a memory 177, a display 178, a user input device 179, a communication interface 180, a picture archiving and communications system (PACS) 181, and a server 182.

The gantry 171 may be configured to support the rotational frame 172, the X-ray source 173, and the X-ray detector 174. The rotational frame 172 may be configured to rotate the X-ray source 173 and the X-ray detector 174 around a subject that is positioned on the table 175. The X-ray source 173 may be configured to emit X-ray radiation in the form of an X-ray beam towards the subject and the X-ray detector 174. The X-ray detector 174 may be configured to detect X-ray radiation emitted by the X-ray source 173 and attenuated by the subject. The table 175 may be configured to support the subject during a scan of the subject. During a scan of the subject, the gantry 171 may rotate the rotational frame 172 around the subject to change an angle at which an X-ray beam emitted by the X-ray source 173 intersects the subject. The X-ray detector 174 may acquire projection data by detecting radiation of the X-ray beam

The processor 176 may be configured to control operations of the preoperative imaging system 170. For example, the processor 176 may be a CPU, a GPU, an APU, a microprocessor, a microcontroller, a DSP, an FPGA, an ASIC, or the like. The processor 176 may be implemented in hardware, firmware, or a combination of hardware and software. The processor 176 may include one or more processors 176 configured to perform the operations described herein. For example, a single processor 176 may be configured to perform all of the operations described herein. Alternatively, multiple processors 176, collectively, may be configured to perform all of the operations described herein, and each of the multiple processors 176 may be configured to perform a subset of the operations described herein. For example, a first processor 176 may perform a first subset of the operations described herein, a second processor 176 may be configured to perform a second subset of the operations described herein, etc.

The processor 176 may be configured to control the gantry 171, movement of the rotational frame 172, the X-ray source 173, the X-ray detector 174, and movement of the table 175. The processor 176 may receive projection data generated during the scan, and generate a medical image using the projection data.

The memory 177 may be configured to store information and/or instructions for use by the processor 176. The memory 177 may be a non-transitory computer-readable medium. For example, the memory 177 may be a RAM, a ROM, a flash memory, a magnetic memory, an optical memory, or the like. The memory 177 may be configured to store instructions that, when executed by the processor 176, cause the processor 176 to perform the operations described herein.

The display 178 may be configured to display information. For example, the display 178 may be a monitor, a light-emitting diode (LED) display, a cathode ray tube, a projector display, a touchscreen, tablet computer, mobile phone, or the like. The display 178 may display medical images in real-time. For example, the display 178 may display the medical images within one second, two seconds, five seconds, etc., of the medical images being generated, a scan being completed, or the like.

The user input device 179 may be configured to receive a user input, and provide the user input to the processor 176. For example, the user input device 179 may be a touch screen display, a keyboard, a keypad, a mouse, a button, a switch, a microphone, or the like. Additionally, or alternatively, the user input device 179 may be configured to sense information. For example, the user input device 179 may sense information from an electro-magnetic positioning system, an inertial measurement system, an accelerometer, a gyroscope, an actuator, or the like.

The communication interface 180 may be configured to enable the processor 176 to communicate with other devices, such as via a wired connection, a wireless connection, or a combination of wired and wireless connections. For example, the communication interface 180 may include an Ethernet interface, an optical interface, a coaxial interface, an infrared interface, a radio frequency (RF) interface, a universal serial bus (USB) interface, a wireless fidelity (Wi-Fi) interface, a cellular network interface, or the like. The PACS 181 may be configured to communicate with external systems and/or networks to permit users at various locations to access the medical image. The server 182 may be configured to store one or more models as described herein. For example, the server 182 may be an on-premises server, a cloud server, a virtual machine, or the like.

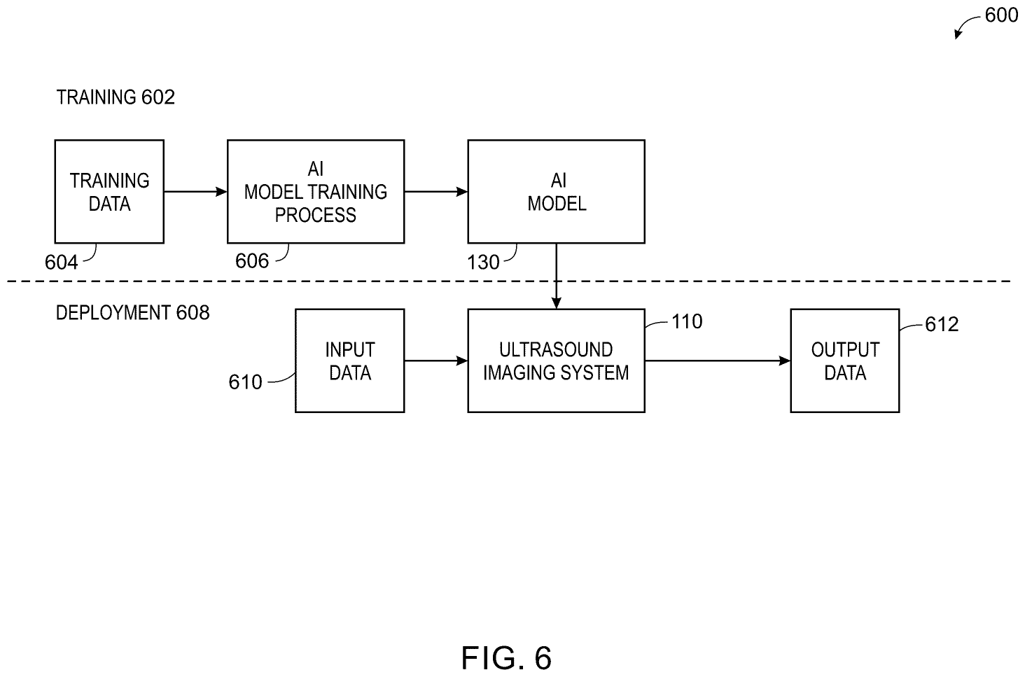

FIG. 6 is a diagram of an example process 600 for training an AI model. As shown in FIG. 6, the process 600 may include a training phase 602 and a deployment phase 608.

In the training phase, a training device may receive and process training data to generate the trained AI model 130 for determining a data quality of ultrasound data. The training data may include a plurality of training datasets respectively including ultrasound data and corresponding data qualities. The training data may be generated, received, or otherwise obtained from internal and/or external resources.

Generally, the AI model 130 may include a set of variables (e.g., nodes, neurons, filters, or the like) that are tuned (e.g., weighted, biased, or the like) to different values via the application of the training data. According to an embodiment, the training process may employ supervised, unsupervised, semi-supervised, and/or reinforcement learning processes to train the AI model 130. According to an embodiment, a portion of the training data may be withheld during training and/or used to validate the trained AI model 130.

For supervised learning processes, the training data may include labels or scores that may facilitate the training process by providing a ground truth. For example, the labels or scores may indicate a data quality corresponding to ultrasound data. Training may proceed by feeding a training dataset into the AI model 130. The AI model 130 may have variables set at initialized values (e.g., at random, based on Gaussian noise, based on pre-trained values, or the like). The AI model 130 may generate an output. The output may be compared with the corresponding label or score (e.g., the ground truth), which may then be back-propagated through the AI model 130 to adjust the values of the variables. This process may be repeated for a plurality of samples at least until a determined loss or error is below a predefined threshold. According to an embodiment, some of the training data may be withheld and used to further validate or test the trained AI model 130.

For unsupervised learning processes, the training data may not include pre-assigned labels or scores to aid the learning process. Instead, unsupervised learning processes may include clustering, classification, or the like, to identify naturally occurring patterns in the training data. As an example, training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data with pre-assigned labels or scores and training data without pre-assigned labels or scores may be used to train the AI model 130.

After being trained, the trained AI model 130 may be stored and subsequently applied by the ultrasound imaging system 110 during the deployment phase 608. For example, during the deployment phase 608, the trained AI model 130 executed by the ultrasound imaging system 110 may receive ultrasound data, and determine a data quality of the ultrasound data.

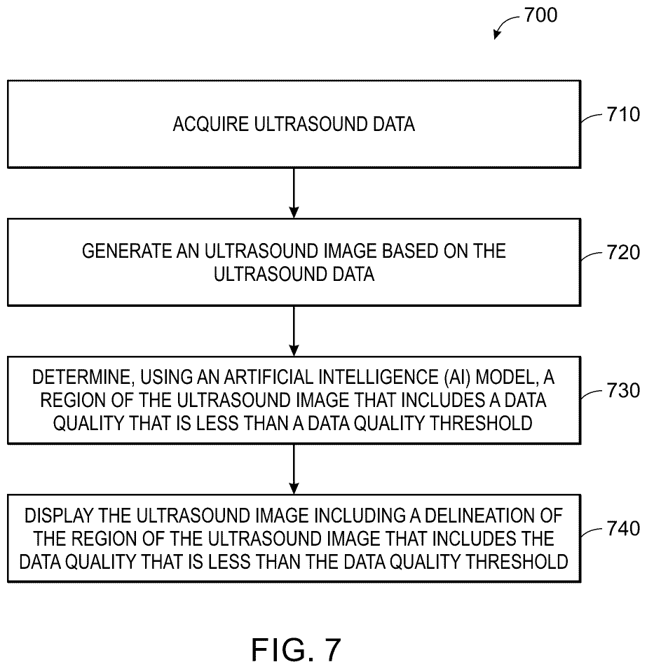

FIG. 7 is a flowchart of an example process 700 for displaying data quality information in association with ultrasound images. For example, the ultrasound imaging system 110 may perform the operations of the process 700. However, in other embodiments, one or more other systems may perform one or more operations of the process 700.

As shown in FIG. 7, the process 700 may include acquiring ultrasound data (operation 710). For example, the ultrasound imaging system 110 may acquire ultrasound data of a region of interest of a subject. The region of interest may be any anatomical structure of the subject. The subject may be a person, an animal, a phantom, or the like.

As further shown in FIG. 7, the process 700 may include generating an ultrasound image based on the ultrasound data (operation 720). For example, the ultrasound imaging system 110 may generate an ultrasound image based on the ultrasound data. The ultrasound image may be a 2D ultrasound image, a 3D ultrasound image, or the like.

As further shown in FIG. 7, the process 700 may include determining, using an AI model, a region of the ultrasound image that includes a data quality that is less than a data quality threshold (operation 730). For example, the ultrasound imaging system 110 may determine, using the AI model 130, a region of the ultrasound image that includes a data quality that is less than a data quality threshold.

As used herein, “data quality” may refer to a measure of a condition of ultrasound data that identifies a fitness of the ultrasound data for a particular use. For example, a data quality of the ultrasound data may identify an accuracy, a completeness, a comprehensiveness, a consistency, a timeliness, a uniqueness, a validity, or the like, of the ultrasound data. The data quality threshold may be a threshold that, if satisfied, indicates that the ultrasound data is accurate, complete, comprehensive, consistent, timely, unique, valid, or the like. Further, the data quality threshold may be a threshold that, if not satisfied, indicates that the ultrasound data is inaccurate, incomplete, incomprehensive, inconsistent, untimely, invalid, or the like. As used herein, “satisfied” may refer to being greater than, being greater than or equal to, being less than, being less than or equal to, or the like.

The ultrasound imaging system 110 may determine a data quality of the ultrasound data. According to an embodiment, the ultrasound imaging system 110 may determine the data quality of the ultrasound data based on a signal quality metric. For example, the signal quality metric may be a signal-to-noise ratio (SNR), a mean squared error (MSE), a bit error rate (BER), or the like. According to an embodiment, the ultrasound imaging system 110 may determine the data quality of the ultrasound data based on an image quality attribute. For example, the image quality attribute may be a sharpness, a noise, a contrast, a distortion, an artifact level, or the like. According to an embodiment, the ultrasound imaging system 110 may determine the data quality of the ultrasound data using the AI model 130. For example, the ultrasound imaging system 110 may input an ultrasound image into the AI model 130, and determine a data quality of the ultrasound image based on an output of the AI model 130. The AI model 130 may be trained using training data that includes known ultrasound images and known data qualities.

The ultrasound imaging system 110 may determine a region of the ultrasound image that includes a data quality that is less than a data quality threshold. For example, the ultrasound imaging system 110 may determine respective data qualities of various portions of the ultrasound image, and compare the respective data qualities to the data quality threshold. A portion of the ultrasound image may be a pixel, a set of pixels, or the like. Further, the ultrasound imaging system 110 may determine a region of the ultrasound image that includes portions of the ultrasound image that have data qualities that do not satisfy the data quality threshold.

As further shown in FIG. 7, the process 700 may include displaying the ultrasound image including a delineation of the region of the ultrasound image that includes the data quality that is less than the data quality threshold (operation 740). As in FIG. 7, a delineation of the entire scanned region may also be desired. For example, the ultrasound imaging system 110 may display the ultrasound image including a delineation of the region of the ultrasound image that includes the data quality that is less than the data quality threshold.

According to an embodiment, the ultrasound imaging system 110 may display the ultrasound image including a delineation that visually delineates the region of the ultrasound image that includes the data quality that is less than the data quality threshold. As an example, the ultrasound imaging system 110 may delineate the region using a visual indicator (e.g., a line, a box, a color, a pattern, or the like). As another example, the ultrasound imaging system 110 may delineate the region by removing the region, by replacing the region, or the like. According to an embodiment, the ultrasound imaging system 110 may display a notification identifying that the region includes the data quality that is less than the data quality threshold.

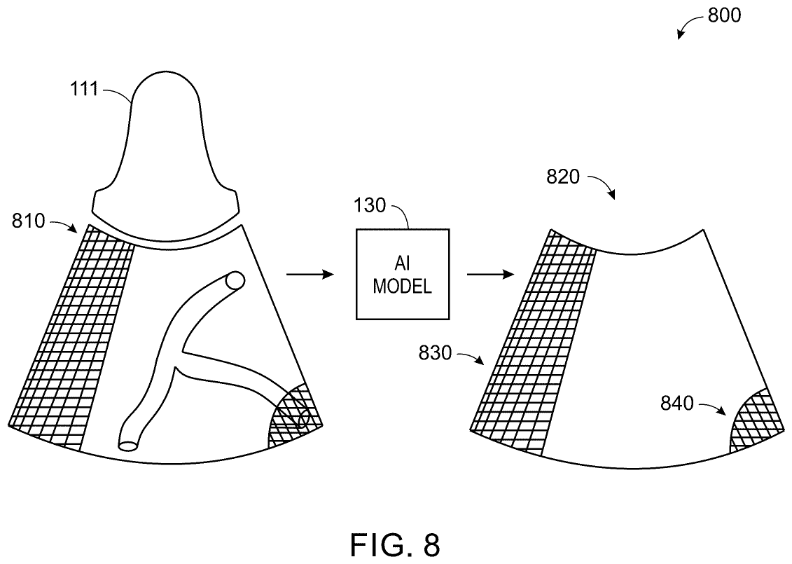

FIG. 8 is a diagram 800 of an example process for displaying data quality information for ultrasound imaging. As shown in FIG. 8, the ultrasound imaging system 110 may input an ultrasound image 810 into the AI model 130, and generate an ultrasound image 820 including a delineation of a region 830 and a region 840 that include data qualities that are less than a data quality threshold based on an output of the AI model 130.

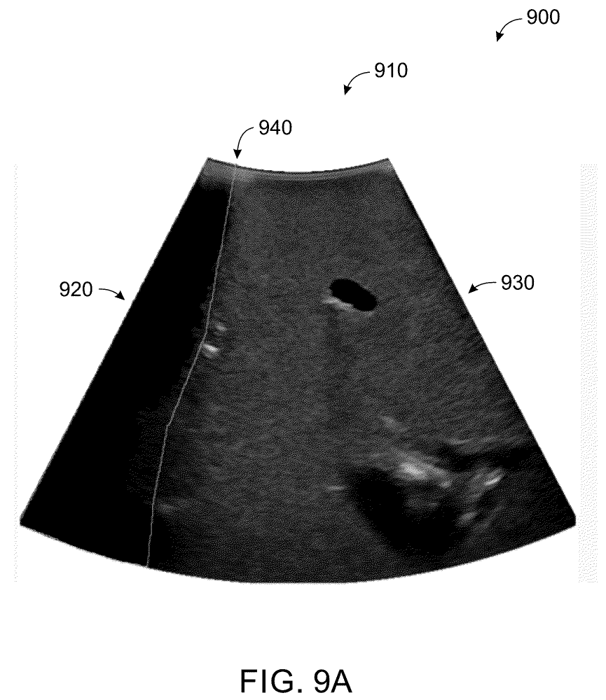

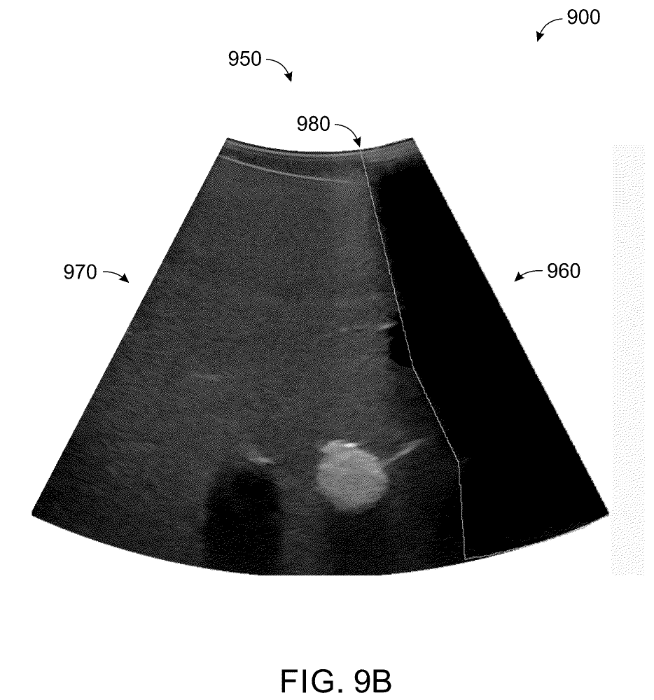

FIGS. 9A and 9B are diagrams 900 of example ultrasound images displaying data quality information. As shown in FIG. 9A, an ultrasound image 910 may include a region 920 that includes a data quality that is less than a data quality threshold, and a region 930 that includes a data quality that is greater than the data quality threshold. Further, the ultrasound image 910 may include a visual indicator 940 that delineates the region 910 includes the data quality that is less than the data quality threshold. As shown in FIG. 9B, an ultrasound image 950 may include a region 960 that includes a data quality that is less than a data quality threshold, and a region 970 that includes a data quality that is greater than the data quality threshold. Further, the ultrasound image 950 may include a visual indicator 980 that delineates the region 960 includes the data quality that is less than the data quality threshold.

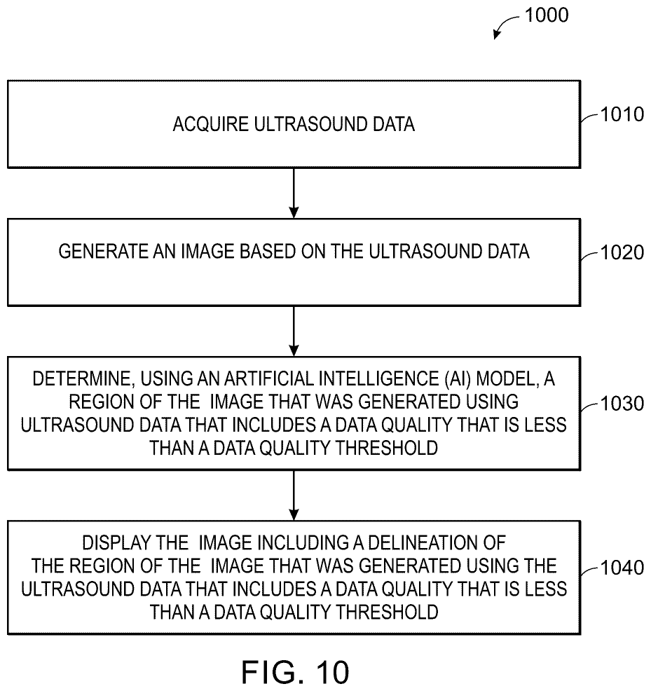

FIG. 10 is a flowchart of an example process 1000 for displaying data quality information in association with 3D images. For example, the ultrasound imaging system 110 may perform the operations of the process 1000. However, in other embodiments, one or more other systems may perform one or more operations of the process 1000.

As shown in FIG. 10, the process 1000 may include acquiring ultrasound data (operation 1010). For example, the ultrasound imaging system 110 may acquire ultrasound data of a region of interest of a subject. The region of interest may be any anatomical structure of the subject. The subject may be a person, an animal, a phantom, or the like.

As further shown in FIG. 10, the process 1000 may include generating an image based on the ultrasound data. For example, the ultrasound imaging system 110 may generate an image based on the ultrasound data. The image may be a 2D image, a 3D image, or the like. The image may be an image of the region of interest. The image may include an anatomical structure of the region of interest. For example, the anatomical structure may be a blood vessel, a tissue, or the like. The ultrasound imaging system 110 may segment the anatomical structure using the ultrasound data, and generate the image based on segmenting the anatomical structure.

As further shown in FIG. 10, the process 1000 may include determining, using an artificial intelligence (AI) model, a region of the image that was generated using ultrasound data that includes a data quality that is less than a data quality threshold (operation 1030). For example, the ultrasound imaging system 110 may determine, using the AI model 130, a region of the 3D image that was generated using ultrasound data that includes a data quality that is less than a data quality threshold.

The ultrasound imaging system 110 may determine the data quality of the ultrasound data used to generate the image. According to an embodiment, the ultrasound imaging system 110 may determine the data quality of the ultrasound data based on a signal quality metric. For example, the signal quality metric may be an SNR, an MSE, or the like. According to an embodiment, the ultrasound imaging system 110 may determine the data quality of the ultrasound data based on an image quality attribute. For example, the image quality attribute may be a sharpness, a noise, a contrast, a distortion, an artifact level, or the like. According to an embodiment, the ultrasound imaging system 110 may determine the data quality of the ultrasound data using the AI model 130. For example, the ultrasound imaging system 110 may input an ultrasound image into the AI model 130, and determine a data quality of the ultrasound image based on an output of the AI model 130.

The ultrasound imaging system 110 may determine a region of the image that was generated using ultrasound data that includes a data quality that is less than a data quality threshold. For example, the ultrasound imaging system 110 may determine respective data qualities of the ultrasound data that was used to generate the image, and compare the respective data qualities to the data quality threshold. Further, the ultrasound imaging system 110 may determine a region of the image that was generated using ultrasound data that includes data qualities that do not satisfy the data quality threshold.

As further shown in FIG. 10, the process 1000 may include displaying the image including a delineation of the region of the image that was generated using the ultrasound data that includes a data quality that is less than a data quality threshold. For example, the ultrasound imaging system 110 may display the image including a delineation of the region of the image that was generated using the ultrasound data that includes a data quality that is less than a data quality threshold.

According to an embodiment, the ultrasound imaging system 110 may display the image including a delineation that visually delineates the region of the image that was generated using the ultrasound data that includes a data quality that is less than a data quality threshold. As an example, the ultrasound imaging system 110 may delineate the region using a visual indicator (e.g., a line, a box, a color, a pattern, or the like). As another example, the ultrasound imaging system 110 may delineate the region by removing the region, by replacing the region, or the like. According to an embodiment, the ultrasound imaging system 110 may display a notification identifying that the region was generated using the ultrasound data that includes a data quality that is less than a data quality threshold.

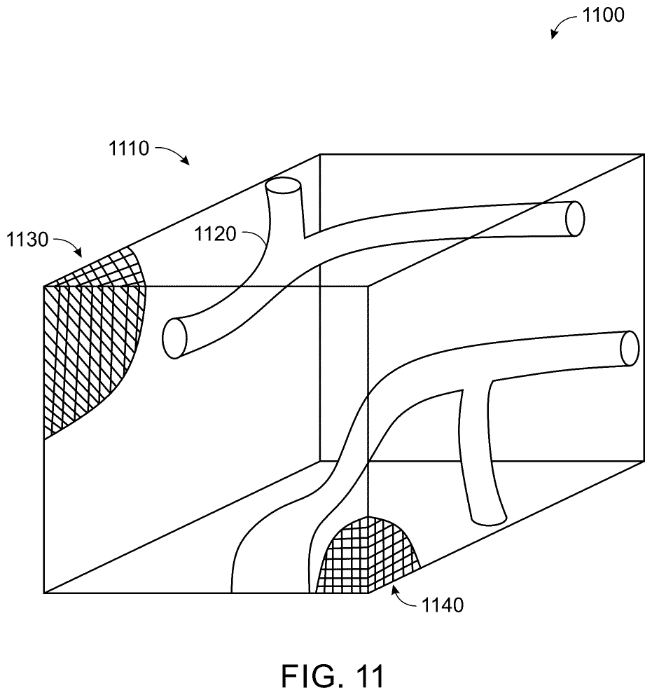

FIG. 11 is a diagram of an example 3D image 1110 displaying data quality information. As shown in FIG. 11, the 3D image 1110 may include an anatomical structure 1120 that was segmented using ultrasound data. Further, as shown, the 3D image 1110 may include a region 1130 and a region 1140 that were generated using ultrasound data that includes a data quality that is less than a data quality threshold.

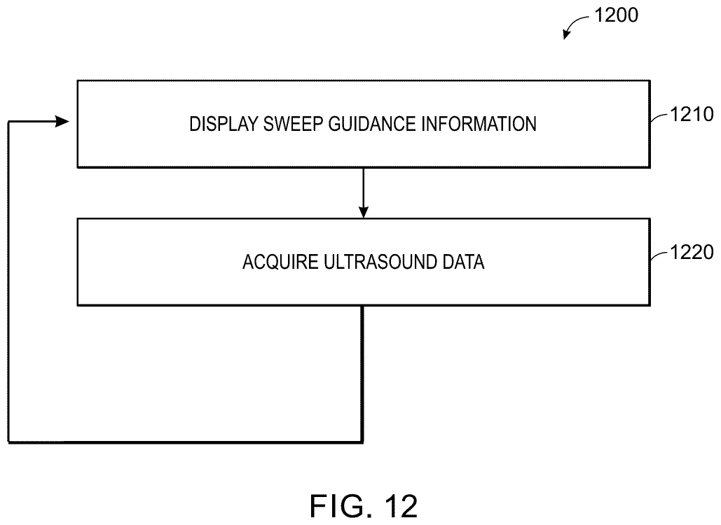

FIG. 12 is a flowchart of an example process 1200 for displaying sweep guidance information for ultrasound data acquisition. For example, the ultrasound imaging system 110 may perform the operations of the process 1200. However, in other embodiments, one or more other systems may perform one or more operations of the process 1200.

As shown in FIG. 12, the process 1200 may include displaying sweep guidance information (operation 1210). For example, the ultrasound imaging system 110 may display sweep guidance information. The sweep guidance information may be information that guides an operator of the ultrasound imaging system 110 to acquire ultrasound data.

According to an embodiment, the sweep guidance information may include a visual indicator that guides a position of the ultrasound probe 111 relative to the subject. For example, the visual indicator may guide the operator to place the ultrasound probe 111 at a particular position relative to the subject. Additionally, or alternatively, the sweep guidance information may include a visual indicator that guides an orientation of the ultrasound probe 111 relative to the subject. For example, the visual indicator may guide the operator to orient the ultrasound probe 111 at a particular orientation relative to the subject. Additionally, or alternatively, the sweep guidance information may include a visual indicator that guides a velocity of the ultrasound probe 111. For example, the visual indicator may guide the operator to move the ultrasound probe 111 at a particular velocity. Additionally, or alternatively, the sweep guidance information may include a visual indicator that guides a direction of movement of the ultrasound probe 111. For example, the visual indicator may guide the operator to move the ultrasound probe 111 at a particular trajectory relative to the subject.

According to an embodiment, the sweep guidance information may include a visual indicator that displays an amount of ultrasound data acquired relative to a region of interest. For example, the visual indicator may display a total amount of ultrasound data to be acquired and an actual amount of acquired ultrasound data. As additional ultrasound data is acquired, the ultrasound imaging system 110 may update the actual amount of acquired ultrasound data.

According to an embodiment, the sweep guidance information may include a visual indicator that displays a 3D image generated based on the ultrasound data. Further, the ultrasound imaging system 110 may update the 3D image as additional ultrasound data is acquired.

According to an embodiment, the ultrasound imaging system 110 may generate the sweep guidance information based on tracking data acquired from the tracking system 150. For example, the ultrasound imaging system 110 may acquire tracking data from the tracking system 150, determine a position, an orientation, a velocity, and/or a movement direction of the ultrasound probe 111 relative to one or more fiducials, and generate the sweep guidance information based on the position, the orientation, the velocity, and/or the movement direction of the ultrasound probe 111 relative to the one more fiducials. The one or more fiducials may include the subject, a region of interest of the subject, an anatomical feature of the subject, an imaging location, or the like.

Additionally, or alternatively, the ultrasound imaging system 110 may generate the sweep guidance information based on preoperative imaging data acquired from the preoperative imaging system 170. For example, the ultrasound imaging system 110 may register ultrasound data acquired by the ultrasound probe 111 with the preoperative imaging data, determine a position, an orientation, a velocity, and/or a movement direction of the ultrasound probe 111 relative to the preoperative imaging data, and generate the sweep guidance information based on the position, the orientation, the velocity, and/or the movement direction of the ultrasound.

According to the embodiment, the ultrasound imaging system 110 may use the information from a guided ultrasound sweep to locally re-align the ultrasound data to the preoperative data for a more accurate registration.

According to an embodiment, the ultrasound imaging system 110 may compare acquired ultrasound data with the preoperative imaging data and determine whether ultrasound data corresponding to an entire dataset of the preoperative imaging data has been acquired. For example, if the preoperative imaging data is of a region of interest, then the ultrasound imaging system 110 may determine whether ultrasound data of the entire region of interest has been acquired. The ultrasound imaging system 110 may generate the sweep guidance based on the comparison to guide the operator of the ultrasound imaging system 110 to acquire missing ultrasound data that corresponds to the acquired preoperative imaging data.

According to an embodiment, the ultrasound imaging system 110 may determine a data quality of acquired ultrasound data and generate the sweep guidance information based on the data quality of the acquired ultrasound data. For example, if the data quality is less than the data quality threshold, then the ultrasound imaging system 110 may generate sweep guidance information that guides the operator of the ultrasound imaging system 110 to acquire ultrasound data having a higher data quality. For instance, the sweep guidance information may guide the operator to re-position and/or re-orient the ultrasound probe 111, move the ultrasound probe 111 more slowly, move the ultrasound probe 111 to re-acquire ultrasound data of a particular region, or the like.

As further shown in FIG. 12, the process 1200 may include acquiring ultrasound data based on displaying the sweep guidance information (operation 1220). For example, the ultrasound imaging system 110 may acquire ultrasound data based on displaying the sweep guidance information. The process 1200 may return to operation 1210. That is, the ultrasound imaging system 110 may display updated sweep guidance information based on the acquired ultrasound data.

According to an embodiment, the ultrasound imaging system 110 may display information that identifies that ultrasound data is to be acquired. For example, the ultrasound imaging system 110 may determine that the ultrasound probe 111 is positioned at a particular positon and/or oriented at a particular orientation, and display information that instructs the operator of the ultrasound imaging system 110 to acquire the ultrasound data. Alternatively, the ultrasound imaging system 110 may determine that the ultrasound probe 111 is positioned at a particular positon and/or oriented at a particular orientation, and automatically acquire the ultrasound data based on the ultrasound probe 111 being positioned at the particular positon and/or oriented at the particular orientation.

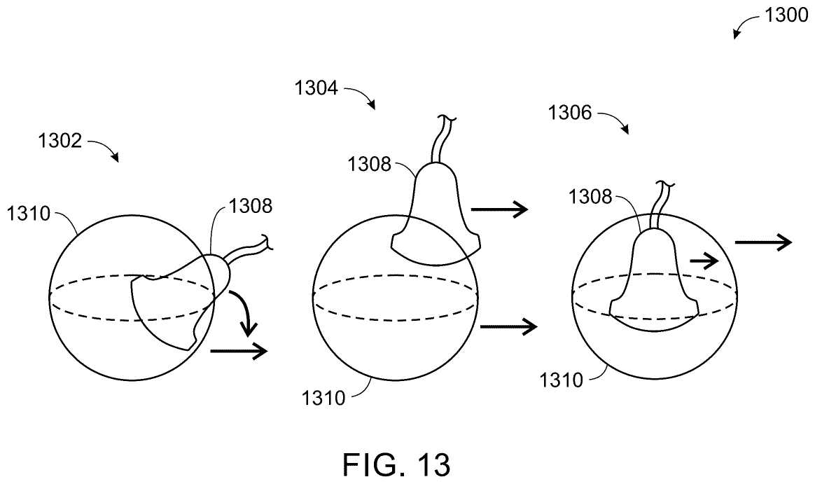

FIG. 13 is a diagram 1300 of sweep guidance information including a visual indicator for guiding an orientation and a velocity of the ultrasound probe 111 during ultrasound data acquisition. As shown by reference numbers 1302, 1304, and 1306, the ultrasound imaging system 110 may display sweep guidance information as the operator of the ultrasound imaging system 110 moves the ultrasound probe 111 relative to the subject. The visual indicator may include a virtual probe 1308 and a sphere 1310. The ultrasound imaging system 110 may adjust the position and/or the orientation of the virtual probe 1308 relative to the sphere 1310 based on the movement of the ultrasound probe 111. If the position, the orientation, and/or the velocity of the ultrasound probe 111 relative to the subject is appropriate for ultrasound data acquisition, then the ultrasound imaging system 110 may display the virtual probe 1308 in the sphere 1310. Alternatively, if the position, the orientation, and/or the velocity of the ultrasound probe 111 relative to the subject is inappropriate for ultrasound data acquisition, then the ultrasound imaging system 110 may display the virtual probe 1308 partially, or entirely, outside of the sphere 1310 depending on the extent of the position, the orientation, and/or the velocity of the ultrasound probe 111 relative to the subject being inappropriate for ultrasound data acquisition.

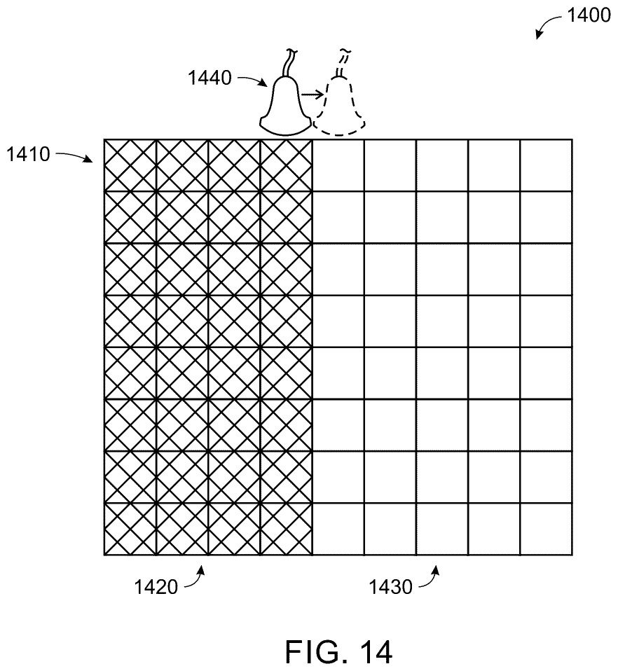

FIG. 14 is a diagram 1400 of sweep guidance information including a visual indicator for guiding a position of the ultrasound probe 111 during ultrasound data acquisition. The visual indicator may include an acquisition volume 1410 that corresponds to a region of interest for which ultrasound data is to be acquired. The acquisition volume 1410 may include an acquired region 1420 that depicts a region of the acquisition volume 1410 for which ultrasound data has been acquired, and a non-acquired region 1430 that depicts a region of the acquisition volume 1410 for which ultrasound data has not yet been acquired. As the operator moves the ultrasound probe 111 to acquire ultrasound data, the ultrasound imaging system 110 may update the display of the acquisition volume 1410 by adding to the acquired region 1420.

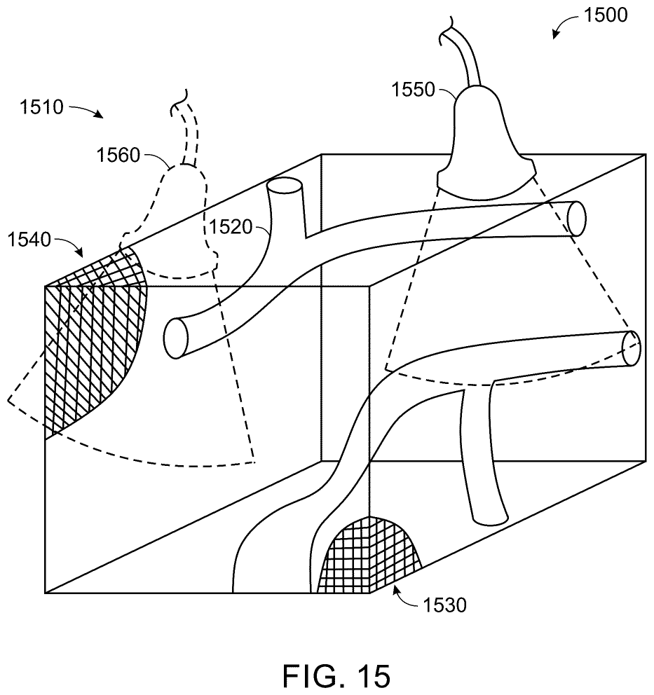

FIG. 15 is a diagram 1500 of sweep guidance information including a visual indicator for guiding a movement of an ultrasound probe 111 to reacquire ultrasound data corresponding to a region of a 3D image that was generated using ultrasound data that includes a data quality that is less than a data quality threshold. The visual indicator may include a 3D image 1510 that displays a segmented anatomical feature 1520, and that displays a region 1530 and a region 1540 that were generated using ultrasound data having a data quality that is less than a data quality threshold. Further, the visual indicator may include a current position 1550 of a virtual probe and a destination position 1560 of the virtual probe. The destination position 1560 may be a position of the ultrasound probe 111 relative to the subject that permits the re-acquisition of ultrasound data corresponding to the region 1530 and the region 1540.

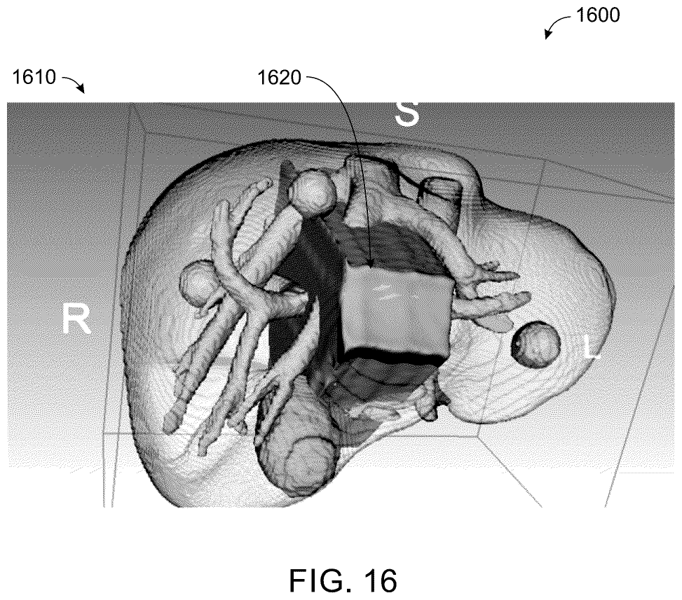

FIG. 16 is a diagram 1600 of sweep guidance information including a visual indicator for guiding a position and an orientation of the ultrasound probe 111 to acquire ultrasound data. The visual indicator may include a 3D image 1610 of a region of interest and include a region 1620 depicting a region where ultrasound data acquisition should take place. According to an embodiment, the ultrasound imaging system 110 may register preoperative imaging data from the preoperative imaging system 170 and ultrasound data from the ultrasound probe 111 tracked by the tracking system 150. The anatomical structures (e.g., blood vessels) of the preoperative imaging data may have previously been segmented. Similarly, the ultrasound imaging system 110 may segment the anatomical structures in the ultrasound data. Because of differences in the organ position during pre-operative scanning and during surgery, the anatomical structures might not match accurately in the two imaging modalities. In that case, the ultrasound imaging system 110 may display sweep guidance information to ensure, or improve the likelihood, that the user sweeps a region that is suitable for accurate alignment between the two modalities. According to an embodiment, the sweep guidance information may guide the user to perform a scan so that the region of insonation includes at least one vessel bifurcation close to a pre-defined target lesion.

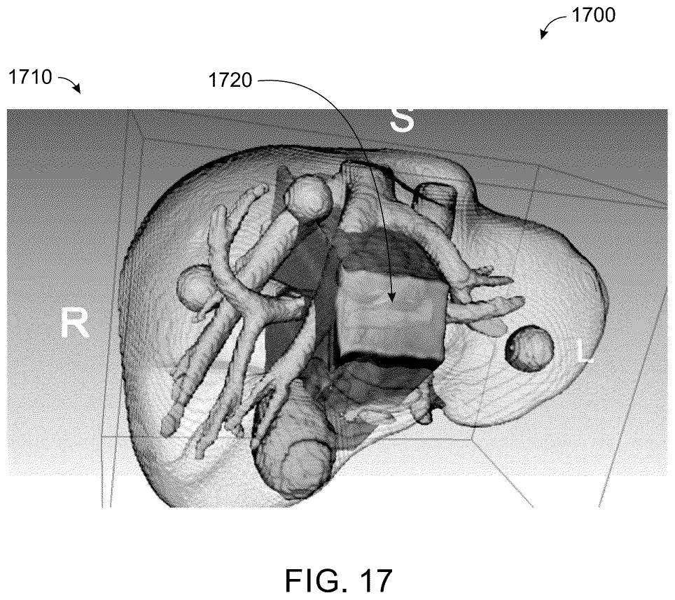

FIG. 17 is a diagram of sweep guidance information including a visual indicator for guiding a position and an orientation of the ultrasound probe to acquire ultrasound data. The visual indicator may include a 3D image 1710 of a region of interest and include a region 1720 depicting a region where ultrasound data acquisition should take place. The region 1720 may be transparent or semi-transparent.

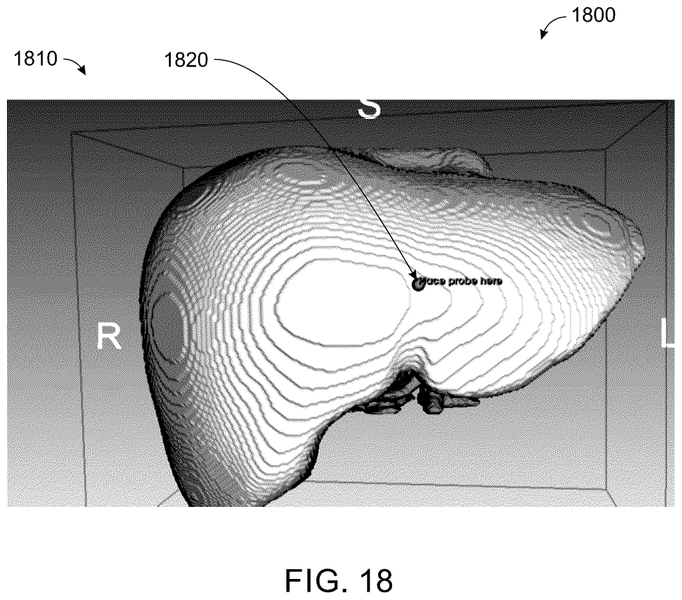

FIG. 18 is a diagram of sweep guidance information including a visual indicator for guiding a position of the ultrasound probe to acquire ultrasound data. The visual indicator may include a 3D image 1810 and a location identifier 1820 for placing the ultrasound probe 111 relative to a region of interest. According to an embodiment, the ultrasound imaging system 110 may register preoperative imaging data from the preoperative imaging system 170 and ultrasound data from the ultrasound probe 111 tracked by the tracking system 150. The anatomical structures (e.g., blood vessels) of the preoperative imaging data may have previously been segmented. Similarly, the ultrasound imaging system 110 may segment the anatomical structures in the ultrasound data. According to an embodiment, the tracking system 150 may track the patient orientation with reasonable accuracy in the ultrasound space, but the position of the ultrasound probe 111 relative to the preoperative imaging data might not be known with reasonable accuracy. In that case, the ultrasound imaging system 110 may display sweep guidance information to guide the user to position the ultrasound probe 111 at a specific anatomical point on the scanning surface which enables the ultrasound data to be registered to the pre-operative data with reasonable accuracy for subsequent processing. The approximate orientation and the approximate position obtained through the tracking system 150 and through the ultrasound probe 111 positioning process enables a subsequent accurate registration of the two imaging modalities.

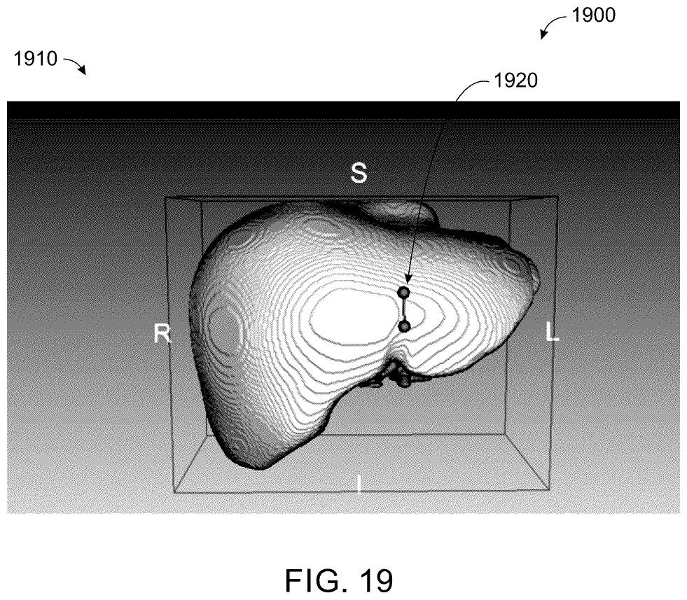

FIG. 19 is a diagram of sweep guidance information including a visual indicator for guiding a position and an orientation of the ultrasound probe to acquire ultrasound data. The visual indicator may include a 3D image 1910 and a location identifier 1920 for placing the ultrasound probe 111 relative to a region of interest and/or moving the ultrasound probe 111 relative to the region of interest.

According to an embodiment, the ultrasound imaging system 110 may register preoperative imaging data from the preoperative imaging system 170 and ultrasound data from the ultrasound probe 111 tracked by the tracking system 150. The anatomical structures (e.g., blood vessels) of the preoperative imaging data may have previously been segmented. Similarly, the ultrasound imaging system 110 may segment the anatomical structures in the ultrasound data. According to an embodiment, the tracking system 150 might not be available such that the patient orientation might not be known with reasonable accuracy in the ultrasound imaging space. Further, the position of the ultrasound probe 111 relative to the preoperative imaging data might not be known with reasonable accuracy. In that case, the ultrasound imaging system 110 may display sweep guidance information to guide the user to position the ultrasound probe 111 at a specific anatomical point on the scanning surface and with a specific orientation such as pointing directly in the superior-inferior direction without roll or yaw which enables the ultrasound data to be registered to the preoperative imaging data with reasonable accuracy for subsequent processing. The approximate orientation and the approximate position obtained through that process enables a subsequent accurate registration of the two imaging modalities.

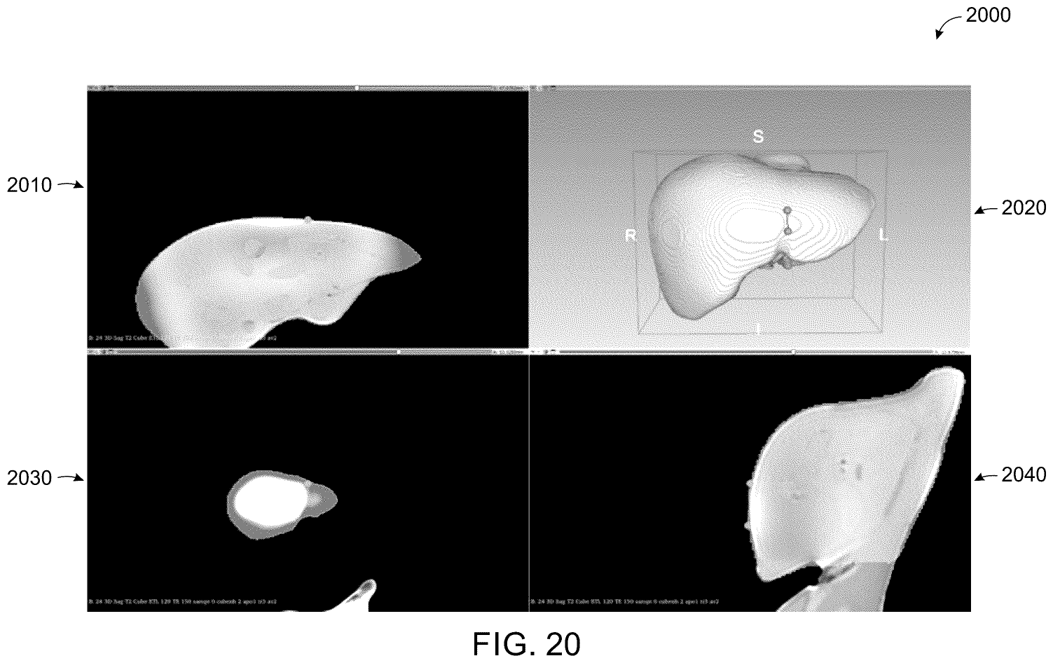

FIG. 20 is a diagram of sweep guidance information including a visual indicator for guiding a position and an orientation of the ultrasound probe to acquire ultrasound data. As shown in FIG. 20, the ultrasound imaging system 110 may display a 3D image 2010, a 3D image 2020, a 3D image 2030, and a 3D image 2040 with relevant regions where ultrasound data acquisition is to take place.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. A device comprising:
a memory configured to store instructions; and
one or more processors configured to execute the instructions to:
acquire ultrasound data of a region of interest of a subject;
generate an image of the region of interest of the subject based on the ultrasound data;
determine, based on a signal quality metric of the ultrasound data, a region of the image that was generated using particular ultrasound data, of the ultrasound data, that includes a data quality that is less than a data quality threshold; and
display the image including a delineation of the region of the image that was generated using the particular ultrasound data, of the ultrasound data, that includes the data quality that is less than the data quality threshold.

2. The device of claim 1, wherein the one or more processors are further configured to:
delineate the region by removing the region from the image.

3. The device of claim 1, wherein the one or more processors are further configured to:
display sweep guidance information including a visual indicator that includes a current position of a virtual probe and a destination position of the virtual probe that guides an operator to re-acquire ultrasound data of the region of the image that was generated using the particular ultrasound data, of the ultrasound data, that includes the data quality that is less than the data quality threshold.

4. The device of claim 1, wherein the one or more processors are further configured to:
display sweep guidance information including a visual indicator that guides an operator to orient an ultrasound probe at an orientation relative to the subject.

5. The device of claim 1, wherein the one or more processors are further configured to:
display sweep guidance information including a visual indicator that guides an operator of an ultrasound probe to move the ultrasound probe at a particular velocity.

6. The device of claim 1, wherein the one or more processors are further configured to:
display sweep guidance information including a visual indicator that guides an operator to move an ultrasound probe at a particular trajectory relative to the subject.

7. The device of claim 1, wherein the one or more processors are further configured to:
display sweep guidance information including a visual indicator that displays an amount of the ultrasound data acquired relative to the region of interest.

8. A method comprising:
acquiring ultrasound data of a region of interest of a subject;
generating an image of the region of interest of the subject based on the ultrasound data;
determining, based on a signal quality metric of the ultrasound data, a region of the image that was generated using particular ultrasound data, of the ultrasound data, that includes a data quality that is less than a data quality threshold; and
displaying the image including a delineation of the region of the image that was generated using the particular ultrasound data, of the ultrasound data, that includes the data quality that is less than the data quality threshold.

9. The method of claim 8, further comprising:
delineating the region by removing the region from the image.

10. The method of claim 8, further comprising:
displaying sweep guidance information including a visual indicator that includes a current position of a virtual probe and a destination position of the virtual probe that guides an operator to re-acquire ultrasound data of the region of the image that was generated using the particular ultrasound data, of the ultrasound data, that includes the data quality that is less than the data quality threshold.

11. The method of claim 8, further comprising:
displaying sweep guidance information including a visual indicator that guides an operator to orient an ultrasound probe at an orientation relative to the subject.

12. The method of claim 8, further comprising:
displaying sweep guidance information including a visual indicator that guides an operator of an ultrasound probe to move the ultrasound probe at a particular velocity.

13. The method of claim 8, further comprising:
displaying sweep guidance information including a visual indicator that guides an operator to move an ultrasound probe at a particular trajectory relative to the subject.

14. The method of claim 8, further comprising:
displaying sweep guidance information including a visual indicator that displays an amount of the ultrasound data acquired relative to the region of interest.

15. A non-transitory computer-readable medium storing instructions that, when executed by one or more processors, cause the one or more processors to:
acquire ultrasound data of a region of interest of a subject;
generate an image of the region of interest of the subject based on the ultrasound data;
determine, based on a signal quality metric of the ultrasound data, a region of the image that was generated using particular ultrasound data, of the ultrasound data, that includes a data quality that is less than a data quality threshold; and
display the image including a delineation of the region of the image that was generated using the particular ultrasound data, of the ultrasound data, that includes the data quality that is less than the data quality threshold.

16. The non-transitory computer-readable medium of claim 15, wherein the instructions further cause the one or more processors to:
delineate the region by removing the region from the image.

17. The non-transitory computer-readable medium of claim 15, wherein the instructions further cause the one or more processors to:
display sweep guidance information including a visual indicator that includes a current position of a virtual probe and a destination position of the virtual probe that guides an operator to re-acquire ultrasound data of the region of the image that was generated using the particular ultrasound data, of the ultrasound data, that includes the data quality that is less than the data quality threshold.

18. The non-transitory computer-readable medium of claim 15, wherein the instructions further cause the one or more processors to:
display sweep guidance information including a visual indicator that guides an operator to orient an ultrasound probe at an orientation relative to the subject.

19. The non-transitory computer-readable medium of claim 15, wherein the instructions further cause the one or more processors to:
display sweep guidance information including a visual indicator that guides an operator of an ultrasound probe to move the ultrasound probe at a particular velocity.

20. The non-transitory computer-readable medium of claim 15, wherein the instructions further cause the one or more processors to:
display sweep guidance information including a visual indicator that guides an operator to move an ultrasound probe at a particular trajectory relative to the subject.
